(19) United States
(12) Patent Application Publication
(10) Pub. No.: US 2008/0058249 A1
(43) Pub. Date: Mar. 6, 2008

(54) Complexes

(75) Inventors: Anders Carlsson, Stockholm (SE); Conny Bogentoft, Hasselby (SE); Mona Stahle, Stockholm (SE); Johan Heilborn, Stockholm (SE)

Correspondence Address: BIRCH STEWART KOLASCH & BIRCH, PO BOX 747, FALLS CHURCH, VA, 22040-0747, US

(73) Assignee: Lipopeptide AB, Stockholm (SE)

(21) Appl. No.: 10/590,548

(22) PCT Filed: Feb. 23, 2005
(86) PCT No.: PCT/SE05/00252

§ 371 Date: Dec. 22, 2006

Related U.S. Application Data
(60) Provisional application No. 60/546,966, filed on Feb. 24, 2004.

(30) Foreign Application Priority Data
Jul. 28, 2004 (SE) 0401942-8

(51) Int. Cl. A61K 38/00; A61P 43/00; C07K 14/505; C07K 14/585; C07K 14/605; C07K 14/62; C07K 7/16; C12N 9/00

(52) U.S. Cl. 514/3; 435/193; 514/12; 514/8; 530/303; 530/307; 530/308; 530/315; 530/350

(57) ABSTRACT

The invention relates to colloidal aqueous solutions of complexes between a charged peptide and a bilayer-forming galactolipid. The colloidal solutions can be used as a drug delivery system for the charged peptide in the treatment of infections, in wound healing and in other diseases.

The present invention refers to colloidal solutions of new complexes between peptides and bilayer-forming galactolipid materials.

### BACKGROUND OF THE INVENTION

A major goal in the pharmacological arts has been the development of methods and compositions to facilitate the specific delivery of therapeutics to the appropriate cells and tissues that would benefit from such treatment, and the avoidance of the general physiological effects of the inappropriate delivery of such agents to other cells or tissues of the body. This is particularly important in the delivery of antimicrobial and antiviral peptide compounds. These compounds typically have immunogenic or cytotoxic effects that damage or destroy uninfected cells as well as infected cells. In addition, certain compounds, drugs or agents are “activated” or chemically modified by an enzymatic or chemical activity specific for infected cells, in which an activated form of the compounds are particularly toxic. Thus, an efficient delivery system which would enable the delivery of such compounds, particularly said “activated” forms thereof, specifically to infected cells would increase the efficacy of treatment, overcome drug resistance, reduce the associated “side effects” of such drug treatments.

Numerous methods for enhancing the activity and the specificity of drug action have been proposed. One method involves linking the therapeutic agent to a ligand which has an affinity for a receptor, expressed on the desired target cell surface. Using this approach antimicrobial and antiviral compounds are intended to adhere to the target cell following formation of a ligand-receptor complex on the cell surface. Entry into the cell could then follow as the result of internalization of ligand-receptor complexes. Following internalization, the antimicrobial or antiviral compounds may then exert therapeutic effects directly on the cell.

### PRIOR ART

U.S. Pat. No. 6,287,590 discloses a method of forming peptide-lipid complexes by co-lyophilisation. In said method one or more lipids and a peptide, respectively, are dissolved in organic solvents, and the two solutions mixed and lyophilized into a powder, which can subsequently be reconstituted in an aqueous solution forming vesicles sometimes resulting in clear solutions.

WO 2004/067025 demonstrate that mixtures consisting of the peptide LL-37, the C-terminal peptide of the human cathelicidin hCAP18, and galactolipids unexpectedly formed stable, clear colloidal solutions at certain weight ratios. Furthermore, it was shown that the in vitro cytotoxicity of LL-37 was reduced when complexed with galactolipids.

WO 95/20944 discloses the use of galactolipid-based liposomes in pharmaceutical applications. This application does not disclose the use of galactolipids in combination with peptides and proteins in general, particularly not for forming complexes in solution, i.e. colloidal solutions, which show improved stability due to complex formation.

### SUMMARY OF THE INVENTION

The present invention is based on the manufacture of stable peptide-polar lipid complexes, where the peptide is associated to the lipid through non-covalent forces. The invention relates to a colloidal solution of the new complexes comprising charged bioactive compounds, such as water-soluble peptides and proteins, and a neutral bilayer-forming galactolipid material in an aqueous medium. More specifically, the present invention refers to the use of new complexes as drug delivery systems for said soluble peptide drugs. The novel drug delivery system retards degradation of the drug, reduces toxicity, prevents adsorption of the drug to non-biological surfaces, and provides for sustained release of the incorporated drug.

### DESCRIPTION OF THE INVENTION

The present invention refers to a peptide-lipid complex in an aqueous solution, which is characterised in that said lipid is a bilayer-forming galactolipid material and that the weight ratio between the peptide and the galactolipid material is 1:5-1:50, with the proviso that the peptide is not LL-37.

According to a preferred embodiment the weight ratio between the peptide and the galactolipid material is 1:10-1:50.

The present invention discloses stable galactolipid-peptide colloidal solutions, where the galactolipid and the peptide form a complex at certain weight ratios. The peptide shall be charged and amphiphilic and have a molecular weight of less than 30 kDa, such as 1-30 kDa, to form a stable complex. A preferred molecular weight of the peptide lies within the range of 2-20 kDa. Preferred peptides or proteins are those containing amino acid residues, which are positively charged. Lysine, arginine, histidine and ornithine are all naturally occurring amino acids, having basic side chains, which are positively charged at pH 7. Synthetic amino acids, which are positively charged at neutral pH are also possible to incorporate in a synthesized peptide, which are also disclosed in the present invention. Furthermore, preferred peptides or proteins are those, which have four or more positively charged amino acids. The charged amino acids should not be consecutive having sequences such as Lys-Arg-Lys-Arg.

Peptides with negative charged amino acids such as aspartic acid, glutamic acid or gamma-carboxy-glutamic acid are also disclosed in the present invention. The negatively charged amino acids should not be consecutive (Asp-Glu-Asp-Glu).

Preferably, the peptide or protein to be combined with the galactolipids is in addition to amphiphilic also surface active. Besides a charged portion the molecule also should have a nonpolar portion. This may give rise to specific secondary structures in aqueous solution, as well as to aggregate formation (self-association) in aqueous solution.

Suitable counterions are acetate, chloride, etc, for a positively charged peptide, and sodium, potassium, ammonium, etc. for a negatively charged peptide.

Examples of peptides and proteins to be used in accordance with the present invention are, for example, those which form secondary structures in aqueous solution, structures such as a-helices, β-pleated sheets and the like.

Antimicrobial peptides are highly charged effector molecules of the innate immune system, which serve to protect the host against potentially harmful microorganisms. They are conserved through evolution and are widespread in nature. In human, only a handful has been identified so far among which the defensins and the human cathelicidin antimicrobial peptide hCAP18 have been implicated in epithelial defence. It has been proposed that cationic peptides interact with microorganisms by binding to their negatively charged surfaces. Specific examples are the cathelicidins including human cationic antimicrobial protein (hCAP18) and its C-terminal peptide LL-37, PR-39, prophenin, indolicidin, the latter which is a 13 residue cationic peptide-amide with a potent antifungal activity.

It is previously known that LL-37 together with galactolipids form colloidal solutions. The present invention demonstrates that other peptides belonging to the cathelicidin family of peptides also form stable colloidal solutions. The galactolipid and the peptide form a complex at certain weight ratios. According to a specific embodiment of the invention the peptide is a cationic antimicrobial peptide having a molecular weight of 2.5-5 kDa (as the free base). Said peptide forms a complex with a galactolipid material at a peptide:galactolipid weight ratio of 1:10-1:27. Preferred peptides are LL-25, LL-26, LL-27, LL-28, LL-29, LL-30, LL-31, LL-32, LL-33, LL-34, LL-35, LL-36, peptides having a sequence of at least 25 amino acids of the N-terminal part of LL-37, and LL-38. Said peptides are described in WO 2004/067025 and the sequences thereof are given below.

!Peptide? Amino acid sequence

| | |
|---|---|
| LL-37 | LLGDFFRKSKEKIGKEFKRIVQRIKDFLRNLVPRTES |
| LL-36 | LLGDFFRKSKEKIGKEFKRIVQRIKDFLRNLVPRTE |
| LL-35 | LLGDFFRKSKEKIGKEFKRIVQRIKDFLRNLVPRT |
| LL-34 | LLGDFFRKSKEKIGKEFKRIVQRIKDFLRNLVPR |
| LL-33 | LLGDFFRKSKEKIGKEFKRIVQRIKDFLRNLVP |
| LL-32 | LLGDFFRKSKEKIGKEFKRIVQRIKDFLRNLV |
| LL-31 | LLGDFFRKSKEKIGKEFKRIVQRIKDFLRNL |
| LL-30 | LLGDFFRKSKEKIGKEFKRIVQRIKDFLRN |
| LL-29 | LLGDFFRKSKEKIGKEFKRIVQRIKDFLR |
| LL-28 | LLGDFFRKSKEKIGKEFKRIVQRIKDFL |
| LL-27 | LLGDFFRKSKEKIGKEFKRIVQRIKDF |
| LL-26 | LLGDFFRKSKEKIGKEFKRIVQRIKD |
| LL-25 | LLGDFFRKSKEKIGKEFKRIVQRIK |
| LL-38 | LLGDFFRKSKEKIGKEFKRIVQRIKDFLRNLVPRTESS |

### A preferred complex according to the invention comprises the peptide LL-25 and a galactolipid material.

Other charged peptides with antibacterial activity are gramicidin S, magainin, cecropin, histatin, hyphancin, cinnamycin, burforin I, parasin I and protamines.

The invention also refers to complexes, wherein the peptide is an apolipoprotein or an apolipoprotein analogue, such as ApoA-I, ApoA-II, ApoA-IV, ApoC-I, ApoC-II, ApoC-III, ApoE. Apo AI, is a single polypeptide with a molecular weight of 28 kDa. Its primary function is to activate LCAT (lecithin-cholesterol acyl transferase) within the HDL (high density lipoprotein) complex, which catalyzes the esterification of cholesterol.

Although there are a number of delivery systems, which have been presented for peptides in general, none has been found to be useful for these cationic peptides or for other highly charged peptides.

Other examples of peptides, which can form a complex according to the invention are insulin, glucagon, erythropoietin, darbepoietin-alpha, and streptokinase.

Peptide hormones, such as motilin are also included in the group of peptides, which can be used according to the invention. Motilin is a 22 amino acid peptide secreted by endocrinocytes in the mucosa of the proximal small intestine. Motilin participates in controlling the pattern of smooth muscle contractions in the upper gastrointestinal tract. Other peptide hormones are somatropin, desmo-pressin, oxytocin, gonadorelin, nafarelin, octreotid, lanreotid, ganirelix, cetrorelix, teriparatid, and salmon calcitonin.

Bilayer is normally meant the lamellar arrangements of polar lipids in water. The acyl chains form the internal hydrophobic part and the polar head-groups the hydrophilic part of the bilayer. Depending on the concentration of said polar lipids in polar solvents, such as water, stable peptide complexes can be formed.

Preferred polar bilayer-forming galactolipid materials to be mixed or formulated with the peptide are those, which are neutral in charge. Especially useful are the digalactosyidiacylglycerols, and other glycolipids, such as the glycosyl ceramides, either natural or synthetic, in which a non-ionic carbohydrate moiety constitutes the polar head-group. As examples of such polar bilayer-forming galactolipids, either of natural or synthetic origin, can be mentioned digalactosyidiacylglycerol or polar lipid mixtures rich in digalactosyldiacylglycerols. Digalactosyldiacylglycerol, DGDG [1,2-diacyl-3-O-(α-D-galactopyranosyl -(1-6)-O-β-D-galactopyranosyl-glycerol], is a class of lipids belonging to the glycolipid family, well known constituents of plant cell membranes. Galactolipids or galactolipid materials, primarily DGDG and DGDG-rich materials, have been investigated and found to be surface active material of interest in industrial applications such as food, cosmetics, and pharmaceutical products. WO 95/20944 describes the use of DGDG-rich material, a “galactolipid material”, as a bilayer-forming material in polar solvents for pharmaceutical, nutritional and cosmetic use.

According to a preferred aspect the galactolipid material is CPL-Galactolipid, a galactolipid material manufactured by LTP Lipid Technologies Provider AB, Sweden. This is a purified galactolipid fraction from oats. The CPL-Galactolipid is today used in dermatological creams and has been shown to be well tolerated, and to have good absorption properties. CPL-Galactolipid is stable at ambient temperature. Based on these data it can be concluded that the complex can be administered topically during long periods of time, for example in wound healing.

The interactions between the charged peptide and the neutral lipid are sufficiently strong to accomplish a stabilization of the peptide and protect it from degradation both in vitro and in vivo through the complex formation. For example, it may be protected from degradation by proteolytic enzymes which may occur in a physiological environment, such as elastase produced in a wound or various proteases and peptidases found elsewhere in an organism, e.g. in the saliva or in the gut. It may also be protected from hydrolytic or any other chemical degradation. However, the interactions are weak enough to release the peptide from the complex once it has been delivered to the site of action. A charged (zwitterionic) phospholipid may lead to too strong electrostatic interactions with the oppositely charged peptide. As a consequence the complexes tend to precipitate, more or less immediately after preparation. If at all possible to formulate and administer, there is then a potential risk of a far too slow or even zero release of the peptide due to the strong forces between the charged phospholipid and the charged peptide.

Thus, the major advantage of the present peptide-galactolipid complexes from a drug delivery point of view is that the galactolipid provides for a physically and chemically stable formulation in vitro, which protects the peptide from a too rapid enzymatic degradation in vivo. A special aspect of the invention therefore refers to the protection of a peptide from degradation in a biological environment by forming a complex with a galactolipid material.

An aqueous solution refers to a solution having physiologically or pharmaceutically acceptable properties regarding pH, ionic strength, isotonicity etc. As examples can be mentioned isotonic solutions of water and other biocompatible solvents, aqueous solutions, such as saline and glucose solutions, as well as mixtures thereof. The aqueous solution can be buffered, such as phosphate-buffered saline, PBS.

A suitable aqueous medium for the complexes is phosphate-buffered saline (PBS; 10 mM sodium phosphate, 150 mM NaCl, pH 7.4). However, any other aqueous solution with comparable ionic strength and appropriate pH may be used or the preparation.

The invention especially refers to a colloidal solution of a complex as previously described, wherein the mean size of said complexes is below 100 nm.

The invention also refers to a colloidal solution of a complex between LL-37 and a bilayer-forming galactolipid material, wherein the mean size of said complexes is below 100 nm. A preferred complex forming said colloidal solution is between LL-37 as a salt and CPL-Galactolipid in a ratio of 1:5-1:50, preferably 1:5-1:20. The size of such a complex will be smaller than the size of the corresponding peptide-fee liposomes formed by the CPL-Galactolipid.

Colloidal solutions are per definition thermodynamically stable, and unlike liposomal dispersions, they do not separate on storing.

The colloidal solution can in addition to the complex comprise pharmaceutically acceptable excipients, such as a preservative to prevent microbial growth in the composition, antioxidants, additional isotonicity agents, colouring agents, stabilising agents such as non-ionic surfactants and hydrophilic polymers, and the like.

According to another aspect the invention also refers to a method of preparing a colloidal solution, which is characterized in the following steps:
(i) weighing of the galactolipid material as a dry, free-flowing powder in an appropriate container, e.g. a flask made of borosilicate glass or polypropylene plastic, to a final concentration of 1 to 5 mg/g, which container allows for a headspace which is equal to or larger than the final volume of the solution;
(ii) selecting an aqueous medium with an ionic strength >100 mM and an appropriate pH, normally in the range of 4 to 10 but preferably around 7;
(iii) weighing of the peptide in another appropriate container, e.g. a flask made of borosilicate glass or polypropylene plastic, and adding the selected aqueous medium to a peptide concentration corresponding to a final weight ratio between the peptide and galactolipid material of 1:5 to 1:50;
(iv) adding the peptide solution (iii) to the dry galactolipid material (i);

1(v) shaking the mixture from (iv) vigorously at room temperature using a suitable shaker at high speed for at least 1 h or until the mixture has become clear; and
(vi) equilibrating the resulting colloidal solution. Said equilibration preferably takes place overnight at a temperature of 2-8° C.

If the mixture under (v) has not become clear after vigorous shaking for 3 h, the procedure is repeated using another weight ratio between the peptide and the galactolipid material, and/or using another aqueous medium with a different ionic strength.

The resulting colloidal solution may be characterized by means of light transmission measurements using a conventional spectrophotometer. Peptide-galactolipid complexes in the proper colloidal state give rise to a high transmission of light (low turbidity). The resulting peptide-galactolipid complexes may also be characterized by means of size measurements using a dynamic light scattering instrument, where normally a mean size of the peptide-galactolipid complexes well below 100 nm is found. The complexes may also be visualized directly using a transmission electron microscope in combination with the cryogenic vitrification technique.

It should be noted that the procedure does not involve the use of ultrasonicators, high-speed mixers (ultra-turrax), high-pressure homogenisers, or other processing equipment, which is a clear advantage from a technical and economical point of view. Furthermore, it does not require heat treatment, which makes it possible to prepare compositions containing heat sensitive bioactive compounds. Finally, and most importantly, the procedure does not involve the use of potentially harmful organic solvents.

The colloidal nature of the composition makes it possible to prepare it aseptically by employing a final sterile filtration step. This is especially advantageous if the composition contains a bioactive molecule which is heat sensitive and thus not possible to heat sterilise.

The colloidal solution of the delivery system of the invention can be used for parenteral administration of biological active peptides, for instance by subcutaneous, intravenous, intraperitoneal, etc. administration.

The colloidal solution can also be administrated by local delivery, such as topical, rectal, mucosal administration. The complex prevents degradation of the bioactive peptide and stabilizes the drug.

The system can also be used to improve oral absorption of said bioactive compound and improve its transport through biological membranes.

### EXAMPLES

### General Procedure

Stable peptide-galactolipid complexes in aqueous solution are for instance formed by the following general procedure: The galactolipid material in an amount of about 60 mg is weighed in a 100 ml glass flask. The peptide in an amount of about 3 mg is dissolved in 30 ml PBS (10 mM sodium phosphate, 150 mM NaCl, pH 7.4) and this solution is added to the galactolipid material. The sample is vigorously shaken, using a suitable shaker at high speed, for 2 h after which the mixture has become almost clear, and is then allowed to equilibrate and settle for about 30 min at room temperature. Optionally, the almost clear solution is subjected to extrusion through a polycarbonate membrane with a pore size of 100 nm or less, in order to remove or reduce the size of large complexes. Alternatively, the almost clear solution is subjected to filtration through a sterile filter with a pore size of 0.22 μm or less, in order to make the solution sterile.

### Example 1

### Preparation of aqueous mixtures comprising a mixture of a cathelicidin-derived peptide and a galactolipid material

The LL-20, LL-25, LL-37 and LL-38 peptides were synthesized using solid phase synthesis with the 9-fluorenylmethoxycarbonyl / tert-butyl strategy. The crude peptides, as the trifluoroacetate salts, were purified by HPLC and finally isolated by lyophilization. The purity was determined by means of HPLC. Analysis of composition of amino acids showed that the relative amounts of each amino acid corresponded with the theoretical values for the respective peptide. The antimicrobial activity of the peptides was tested using an inhibition assay.

To prepare the solution of the complex the peptide and CPL-Galactolipid are weighed in a 100 ml glass flask and then PBS (10 mM sodium phosphate, 150 mM NaCl, pH 7.4) is added. The sample is vigorously shaken, using a suitable shaker at high speed, for 1-2 h or until the mixture has become clear, and is then allowed to equilibrate and settle for about 30 min at room temperature. Samples of LL-20, LL-25, LL-37 and LL-38 as trifluoroacetate salts and CPL-Galactolipid were prepared using the amounts stated in Table 1 below. The peptide mixtures all contained 0.20% CPL-Galactolipid.

TABLE 1

| | LL-20 | | | | |
|---|---|---|---|---|---|
| | (MW | LL-25 | LL-37 | LL-38 | |
| | 2440 | (MW | (MW | (MW | |
| Sample No | Da) | 3065 Da) | 4493 Da) | 4598 Da) | Appearance |
| 1 | — | — | — | — | Turbid |
| (CPL- | | | | | dispersion |
| Galactolipid | | | | | |
| in PBS) | | | | | |
| 2 | 52 ppm | — | — | — | Cloudy |
| | | | | | dispersion |
| 3 | 98 ppm | — | — | — | Cloudy |
| | | | | | dispersion |
| 4 | — | 68 ppm | — | — | Almost clear |
| | | | | | solution |
| 5 | — | 98 ppm | — | — | Almost clear |
| | | | | | solution |
| 6 | — | — | 44 ppm | — | Slightly |
| | | | | | turbid |
| | | | | | dispersion |
| 7 | — | — | 98 ppm | — | Almost clear |
| | | | | | solution |
| 8 | — | — | 213 ppm | — | Clear |
| | | | | | solution |
| 9 | — | — | — | 50 ppm | Slightly |
| | | | | | turbid |
| | | | | | dispersion |
| 10 | — | — | — | 102 ppm | Almost clear |
| | | | | | solution |

Ocular inspections were made after 2 h and 2 days of storage at room temperature of the mixtures. These inspections revealed that LL-25, LL-37 and LL-38 in the concentration ranges of 68-98 ppm, 98-213 ppm and 50-102 ppm, respectively, resulted in clear or almost clear solutions. The LL-20 mixture showed large sediments in the investigated concentration range. The molecular weight of LL-20 was calculated to be 2.4 kDa.

The interactions between the charged antimicrobial peptide and the neutral lipid are supposed to be sufficiently strong to accomplish a stabilization of the peptide but weak enough to release the peptide from the complex once it has been delivered to the site of action as shown in wound healing experiments.

The data thus demonstrate that stable complexes are formed between the cationic peptide and CPL-Galactolipid only if the peptide has a molecular weight >2.5 kDa. A preferred peptide:galactolipid weight ratio can be 1:10-1:27.

### Example 2

Test of antimicrobial activity of LL-20 and LL-25 complexes The antimicrobial activity was tested using an inhibition zone assay. As a test bacterium, Bacillius megaterium was used. The following data was obtained.

TABLE 2

| Sample | | |
|---|---|---|
| No. | | Mean (mm) |
| 1 | 0.203% CPL-Galactolipid (GL) | Neg |
| | in PBS | |
| 2 | 68 ppm LL-25 + 0.200% GL in | 7.5 |
| | PBS | |
| 3 | 98 ppm LL-25 + 0.200% GL in | 7.9 |
| | PBS | |
| 4 | 52 ppm LL-20 + 0.203% GL in | Neg |
| | PBS | |
| 5 | 98 ppm LL-20 + 0.200% GL in | Neg |
| | PBS | |
| 6 | 100 ppm LL-25 in PBS | 8.8 |

The data shows that LL-25 showed an antimicrobial activity at a concentration of 68 ppm. It was also shown that LL-25 exhibit activity using the complex with CPL-Galactolipid. The complex with LL-20 had no antimicrobial activity.

### Example 3

### Test of enzymatic degradation of LL-37 —a comparative study

From a drug development point of view it would be advantageous if the enzymatic degradation of a peptide could be hampered or blocked since this would increase the half-life of the intact peptide, which then could exert its biological functions over an extended period of time.

Pseudomonas aeruginosa is a common wound pathogen that produces elastase, a hydrolytic enzyme, with capacity to rapidly degrade antimicrobial peptides produced by an infected host, in its efforts to combat bacterial infections. In humans, LL-37 is the most important antimicrobial peptide and its degradation by elastase from Pseudomonas aeruginosa has been studied previously (A. Schmidtchen et al., Molecular Microbiology (2002) 46 (1), 157-168).

In this study we compared the enzymatic degradation rate of LL-37 in an aqueous buffered system to that of a colloidal solution of LL-37 in a galactolipid complex. The experimental procedures for enzymatic degradation were essentially as described using a reverse phase HPLC system (C-18) operating at 210 nm.

In brief: Two stock solutions A and B were prepared. Solution A, “the reference”, contained 100 μg/ml of LL-37 in PBS, pH 7.4. Solution B, “the complex”, contained in addition to 100 μg/ml LL-37 in PBS, pH 7.4, 0.2% of galactolipids (w/w). Two sets of samples were prepared in Eppendorf tubes, 8 tubes from each stock solution. One tube in each set of samples was kept as a negative control (no enzyme added) and to the remaining samples were added an effective amount of elastase from Psuedomonas aeruginosa, giving a final ratio of enzyme to substrate (peptide) of approximately 1:2500. The reactions were kept at 37° C. and samples were withdrawn at predetermined intervals. The reactions were stopped by heating the samples to 100° C. for 5 minutes. After stopping, the reactions were stored at −18° C. prior analysis.

All samples were analysed in duplicates and the peak area for LL-37 was normalized to that of the negative control, which was set to 1.00. Retention times are given as relative retention time (RRT) to LL-37, which is set to RRT=1.00. During degradation of LL-37 in the buffered solution, several different peaks, representing fragments of LL-37 were detected. All fragments eluted at shorter retention times to that of LL-37, indicating their lower molecular weights. From chromatography it is evident that the degradation of LL-37 in a buffered aqueous solution is fast and that the peptide degrades in a step wise manner, first giving a relatively large fragment, at RRT=0.88, which is further degraded to a fragment at RRT=0.66. After 20 h, almost all material had been degraded to low molecular weight fragments having short retention times in the chromatographic system. However, when LL-37 is in the form of a galactolipid complex no detectable amounts of degradation products were found in any of the samples. The results are given in Table 3 below.

TABLE 3

| | | LL-37 in | | | LL-37 in | | |
|---|---|---|---|---|---|---|---|
| | | aqueous buffer | | | galactolipid complex | | |
| | | RRT | RRT | RRT 1.00 | | | RRT 1.00 |
| Time | | 0.66 | 0.88 | (LL-37) | RRT 0.66 | RRT 0.88 | (LL-37) |
| 0 | | — | — | 1.00 | — | — | 1.00 |
| 1 | min | 0.1 | 0.4 | 0.3 | n.d | n.d | 1.00 |
| 5 | min | 0.14 | 0.36 | 0.23 | n.d | n.d | 1.00 |
| 15 | min | 0.27 | 0.04 | n.d. | n.d | n.d | 1.00 |
| 30 | min | 0.54 | n.d | n.d. | n.d | n.d | 1.00 |
| 1 | h | 0.46 | n.d. | n.d. | n.d | n.d | 1.00 |
| 4 | h | 0.31 | n.d. | n.d. | n.d | n.d | 1.00 |
| 20 | h | 0.08 | n.d | n.d. | n.d | n.d | 1.00 |

n.d. = not detected
Only major degradation products are reported.

From the table above it is evident that LL-37 in a buffered aqueous solution is rapidly degraded when treated with elastase from Pseudomonas aerogunosa. However, when LL-37 in the form of a galactolipid complex is subjected to identical experimental conditions no such degradation is observed, clearly demonstrating the protective effect of the galactolipid formulation.

The present invention is not limited in scope by these described examples. It is thus anticipated that it should be possible to form similar complexes based on galactolipids using other bioactive compounds having molecular weights less than 30 kDa, and being amphiphilic with a net charge. The optimal conditions, that is, the weight ratio of peptide to galactolipid material and the total concentration of the two ingredients in the solution can be obtained by experiments. The aqueous solution should have an appropriate composition, ionic strength and pH as described above. The best composition for each unique peptide and galactolipid mixture is thus established and validated by means of the technically simple procedure described above.

1. A peptide-lipid complex in an aqueous solution, characterised in that said lipid is a bilayer-forming galactolipid material and that the weight ratio between the peptide and the galactolipid material is 1:5-1:50, with the proviso that the peptide is not LL-37.

2. The complex according to claim 1, wherein the weight ratio between the peptide and the galactolipid material is 1:10-1:50.

3. The complex according to claim 1 or 2, wherein said peptide is a charged and amphiphilic peptide having a molecular weight below 30 kDa.

4. The complex according to any of claims 1-3, wherein the peptide has at least four positively charged amino acids.

5. The complex according to any of claims 1-4, wherein the peptide is in the form of a pharmaceutically acceptable salt.

6. The complex according to any of claims 1-5, wherein the galactolipid material is a polar lipid mixture rich in digalactosyldiacylglycerols.

7. The complex according to any of claims 1-6, wherein the galactolipid material is CPL-Galactolipid.

8. The complex according to any of claims 1-7, wherein the peptide is an apolipoprotein or an apolipoprotein analogue.

9. The complex according to any of claims 1-7, wherein the peptide is selected from the group consisting of insulin, glucagon, erythropoietin, darbepoietin, streptokinase, somatropin, desmopressin, oxytocin, gonadorelin, nafarelin, octreotid, lanreotid, ganirelix, cetrorelix, teriparalid, and salmon calcitonin.

10. The complex according to any of claims 1-7, wherein the peptide is selected from the group consisting of magainin 2, cecropin, and histatin.

11. The complex according to any of claims 1-7, wherein said peptide is a cationic antimicrobial peptide having a molecular weight of 2.5-5 kDa.

12. The complex according to any of claims 1-7 and 11, having a peptide:galactolipid weight ratio of 1:10-1:27.

13. The complex according to any of claims 1-7, 11 and 12, wherein the peptide is selected from the group consisting of LL-25, LL-26, LL-27, LL-28, LL-29, LL-30, LL-31, LL-32, LL-33, LL-34, LL-35, LL-36, and LL-38.

14. The complex according to any of claims 1-7, and 11-13, comprising the peptide LL-25 and a galactolipid material.

15. A colloidal solution of a complex according to any of claims 1-14, wherein the mean size of said complexes is below 100 nm.

16. A colloidal solution of a complex between LL-37 and a bilayer-forming galactolipid material, wherein the mean size of said complexes is below 100 nm.

17. A colloidal solution of a complex according to claim 15 or 16 for use as a medicament.

18. A method of preparing a colloidal solution according to claim 15 or 16, characterized in the following steps:
(i) weighing of the galactolipid material as a dry, free-flowing powder in an appropriate container, e.g. a flask made of borosilicate glass or polypropylene plastic, to a final concentration of 1 to 5 mg/g, which container allows for a headspace which is equal to or larger than the final volume of the solution;
(ii) selecting an aqueous medium with an ionic strength >100 mM and an appropriate pH, normally in the range of 4 to 10 but preferably around 7;
(iii) weighing of the peptide in another appropriate container, e.g. a flask made of borosilicate glass or polypropylene plastic, and adding the selected aqueous medium to a peptide concentration corresponding to a final weight ratio between the peptide and galactolipid material of 1:5 to 1:50;
(iv) adding the peptide solution (iii) to the dry galactolipid material (i);
(v) shaking the mixture from (iv) vigorously at room temperature using a suitable shaker at high speed for at least 1 h or until the mixture has become clear; and
(vi) equilibrating the resulting collodial solution.

19. Use of a colloidal solution of a complex according to any of claims 1-16 for the manufacture of a medicament.

20. Use of a colloidal solution of a complex according to claim 15 or 16 for the manufacture of a medicament for treatment of infections, wound healing or other diseases with a deficiency in antimicrobial activity.

21. Use of a colloidal solution of a complex according to claim 20 for the manufacture of a medicament for topical treatment of infections, wounds, atopic eczema and other conditions deficient in antimicrobial activity and/or angiogenesis.
